(19) United States
(12) Patent Application Publication
(10) Pub. No.: US 2007/0167909 A1
(43) Pub. Date: Jul. 19, 2007

(54) Disengaging device for a needle hub of a safety syringe

(75) Inventors: Chien-Yuan Lee, Tamsui Town (TW); Chih-Cheng Kao, Taipei (TW)

Correspondence Address: COOPER & DUNHAM, LLP, 1185 AVENUE OF THE AMERICAS, NEW YORK, NY, 10036, US

(73) Assignee: Linfair Engineering (HK) Co., Ltd.

(21) Appl. No.: 11/331,907

(22) Filed: Jan. 16, 2006

(51) Int. Cl. A61M 5/00

(52) U.S. Cl. 604/110

(57) ABSTRACT

A disengaging device for a needle hub of a safety syringe has a seat. The seat is mounted on an annular shoulder of the needle hub and selectively engages with an annular flange on an outer barrel of the safety syringe. Accordingly, the seat can fit with different types of needle hub, and the disengaging device is versatile in use.

### BACKGROUND OF THE INVENTION

1. Field of the Invention

The present invention relates to a disengaging device, and more particularly to a disengaging device for a needle hub of a safety syringe so that safety syringe manufacturers are able to easy to assemble safety syringes.

2. Description of Related Art

For long time, medical wastes are always big problems to environment and people, and more particularly to used syringes. When a person is injured by used syringes, the person may be infected with a fatal disease. To prevent the used syringes from injuring any person, safety syringes are wildly used in hospitals or any places where perform medical procedures.

With reference to FIGS. 16 and 17, a conventional safety syringe comprises an inner barrel (50), a plunger (52), a retractor (54), a needle hub (55) and an outer barrel (51).

The inner barrel (50) selectively hold liquid medication and has a front opening and a rear opening.

The plunger (52) has a front and a stopper (53). The stopper (53) is mounted on the front of the plunger (52) and slidably in the inner barrel (50) through the rear opening. When the plunger (52) is pushed into the inner barrel (50), the stopper (53) pushes medication out of the inner barrel (50) from the front opening.

The retractor (54) is mounted removably on the front opening of the outer barrel (51) and has an outer edge, multiple resilience fingers (541) and a front. The fingers (541) are formed on and radially from the outer edge around the retractor (54).

The needle hub (55) is mounted on the front of the retractor (54) and has a front end and a needle (551). The needle (551) is mounted on and protrudes longitudinally from the front end of the needle hub (55).

The outer barrel (51) has an inner surface, an annular detent (511), a front opening and a rear opening. The rear opening of the outer barrel (51) allows the inner barrel (50) with the needle (551) to be inserted into the outer barrel (51). The front opening of the outer barrel (51) allows the needle to extend out of the outer barrel (51). The annular detent (511) is formed on the inner surface of the outer barrel (51) near the rear opening and selectively engages with the fingers (541) on the retractor (54).

After medication is injected into a patient, the inner barrel (50) is pulled out of the outer barrel (51). The fingers (541) on the retractor (54) engage with the annular detent (511) in the outer barrel (51). When the inner barrel (50) and the outer barrel (51) are separated, the retractor (54) with the needle (551) is held inside the outer barrel (5 1) with the engagement between the annular detent (511) and the fingers (541). The needle (551) cannot be used again and cannot injure anyone when the needle is held inside the outer barrel (51).

However, most safety syringe manufacturers assemble the retractors (54) and the outer barrels (51) with conventional syringes to achieve safety syringes. Because the different syringes have different usage so that sizes of the syringes are quite different. The safety syringe manufacturers have to make different retractors (54) with different sizes to fit with the needle hubs (55) on different syringes so that to assemble and to manufacture a conventional safety syringe is troublesome and costly.

To overcome the shortcomings, the present invention provides a disengaging device for a needle hub of a safety syringe to obviate or mitigate the aforementioned problems.

### SUMMARY OF THE INVENTION

The primary objective of the present invention is to provide a disengaging device for a needle hub of a safety syringe to make safety syringe manufacturers easily and conveniently assemble a safety syringe with the disengaging device.

The disengaging device has a seat. The seat is mounted on an annular shoulder of needle hub and selectively engages with an annular flange of an outer barrel of the safety syringe so that the locking hub with a needle is kept inside the outer barrel after the syringe is retracted from the outer barrel.

Other objectives, advantages and novel features of the invention will become more apparent from the following detailed description when taken in conjunction with the accompanying drawings.

### BRIEF DESCRIPTION OF THE DRAWINGS

FIG. 1 is an exploded view of a safety syringe with a first embodiment of a disengaging device in accordance with the present invention;

FIG. 2 is an operational side view in partial section of the safety syringe with the disengaging device in FIG. 1 showing that the inner barrel is held inside of the outer barrel;

FIG. 3 is an operational side view in partial section of the safety syringe with the disengaging device in FIG. 1 showing that the inner barrel is pulled out from the outer barrel;

FIG. 4 is an operational side view in partial section of the safety syringe with the disengaging device in FIG. 1 showing that the needle hub engages with the outer barrel;

FIG. 5 is an operational side view in partial section of the safety syringe with the disengaging device in FIG. 1 showing that the outer barrel and the inner barrel are separated from each other;

FIG. 6 is an exploded perspective view of another embodiment of a safety syringe with the first embodiment of the disengaging device in accordance with the present invention;

FIG. 7 is an exploded perspective view of a further embodiment of a safety syringe with the first embodiment of the disengaging device in accordance with the present invention;

FIG. 8 is an exploded perspective view of a safety syringe with a second embodiment of a disengaging device in accordance with the present invention;

FIG. 9 is an exploded perspective view of another embodiment of a safety syringe with the second embodiment of the disengaging device in accordance with the present invention;

FIG. 10 is a an exploded perspective view of a safety syringe with a third embodiment of the disengaging device in accordance with the present invention;

FIG. 11 is an exploded perspective view of another embodiment of a safety syringe with the third embodiment of the disengaging device in accordance with the present invention;

FIG. 12 is an exploded perspective view of a safety syringe with a fourth embodiment of the disengaging device in accordance with the present invention;

FIG. 13 is an exploded perspective view of a safety syringe with a fifth embodiment of the disengaging device in accordance with the present invention;

FIG. 14 is an exploded perspective view of a safety syringe with a sixth embodiment of the disengaging device in accordance with the present invention;

FIG. 15 is an exploded perspective view of another embodiment of a safety syringe with the sixth embodiment of the disengaging device in accordance with the present invention;

FIG. 16 is an exploded perspective view of a conventional safety syringe in accordance with the prior art; and

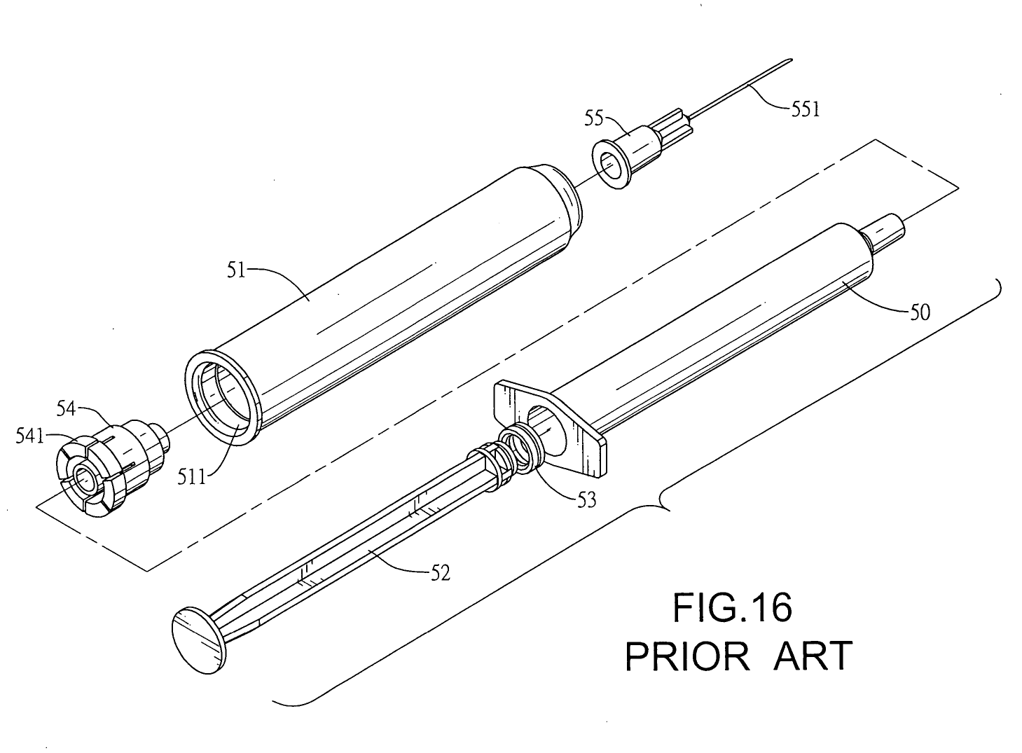

FIG. 17 is a side view in partial section of the safety syringe in FIG. 16 showing that the retractor engages with the outer barrel.

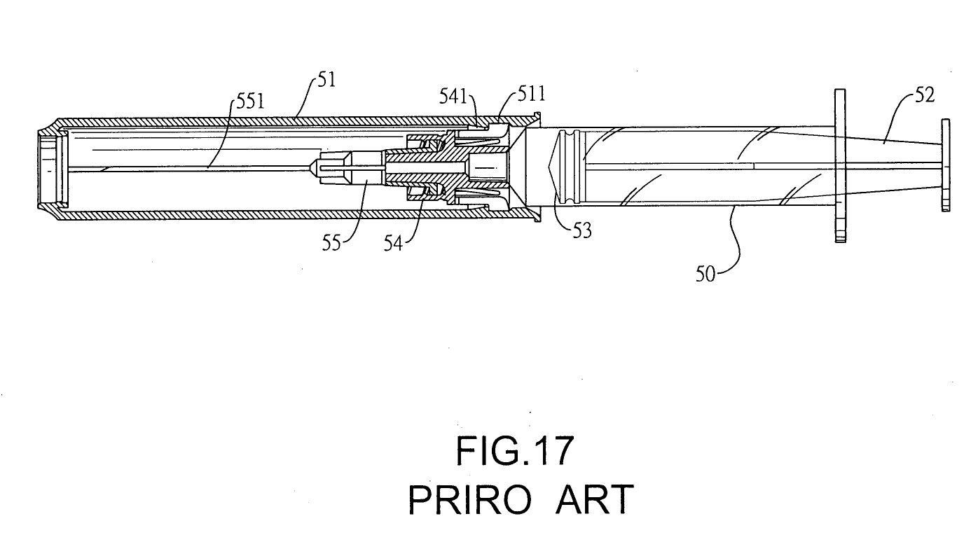

### DETAILED DESCRIPTION OF THE PREFERRED EMBODIMENT

With reference to FIGS., 1,6, 7, 8, 9, 10, 11, 12 and 14, a syringe comprises an inner barrel (10)(10A), a plunger (11), a needle hub (30)(30A) and an outer barrel (20)(20A)(20B)(20C)(20D).

The inner barrel (10)(10A) has a front opening and a rear opening. The plunger (11) is slidably inserted into the inner barrel (20)(20A) through the rear opening. The needle hub (30)(30A) is mounted on the front opening of the inner barrel (10)(10A) and has a front end, a rear end, a needle (31)(31A) and an annular shoulder (32)(32A). The needle (31)(31A) is mounted on the front end of the needle hub (30)(30A). The annular shoulder (32)(32A) is formed at and radially extends from the rear end of the needle hub (30)(30A).

The outer barrel (20)(20A)(20B)(20C)(20D) allows the inner barrel (10)(10A) with the needle (31)(31A) to be inserted into the outer barrel (20)(20A)(20B)(20C)(20D).

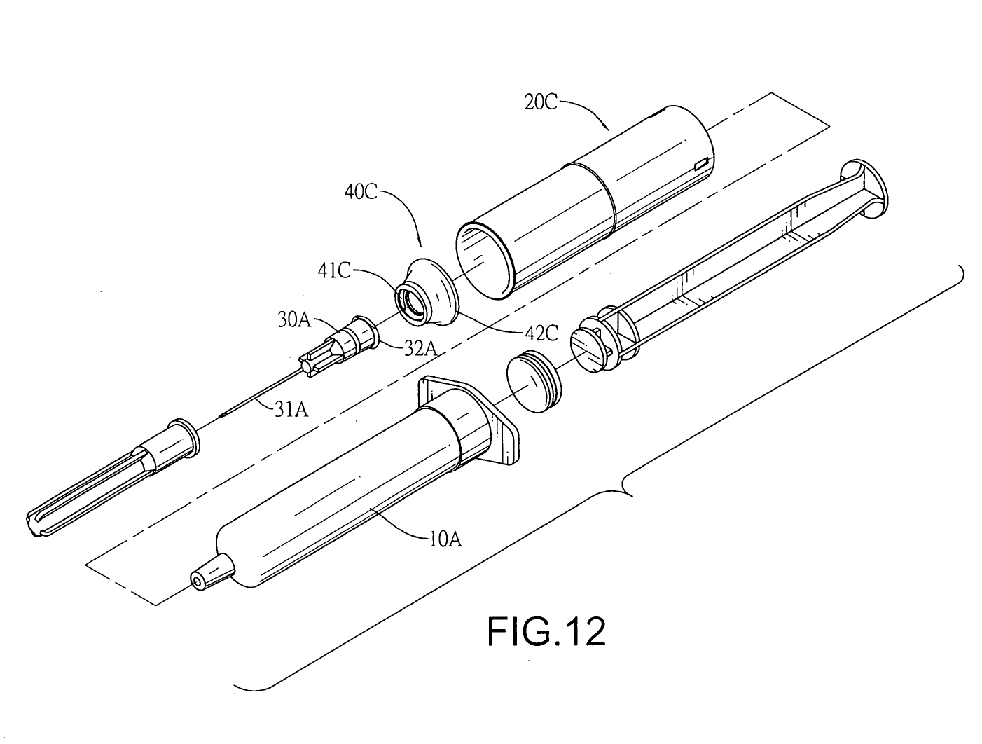

With further reference to FIGS. 12, 13 and 15, the outer barrel (20C) may be tubular and has a front opening, a rear opening, an inner surface, an annular detent and an outer surface. The annular detent is formed at the inner surface of the outer barrel (20C) and near the rear opening of the outer barrel (20C).

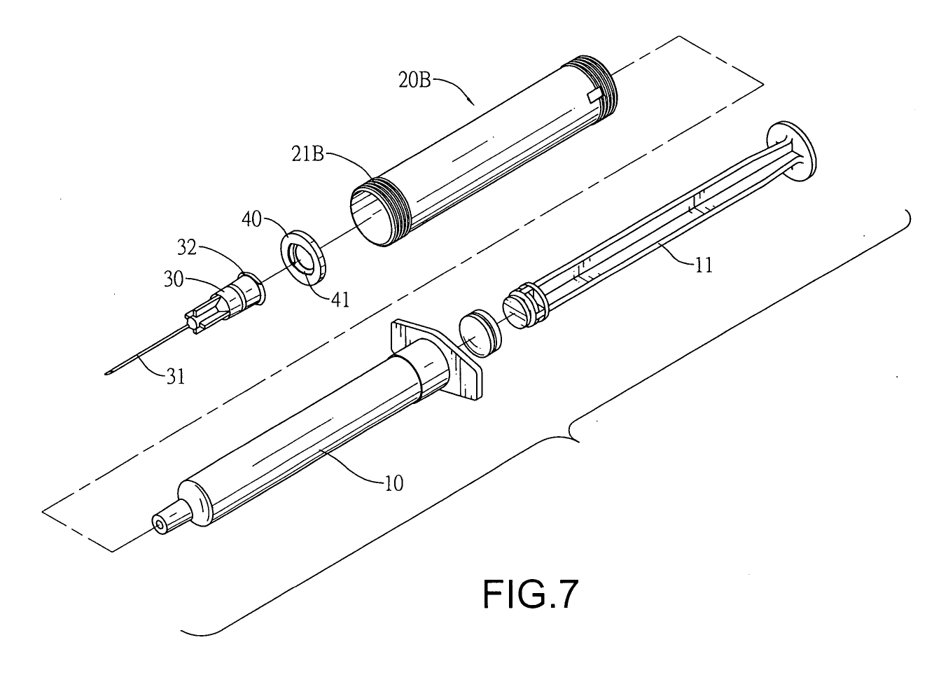

With further reference to FIGS. 7, 9 and 11, the outer barrel (20B) may further have annular grips. The annular grips are formed on the outer surface of the outer barrel (20B) near the front opening and the rear opening.

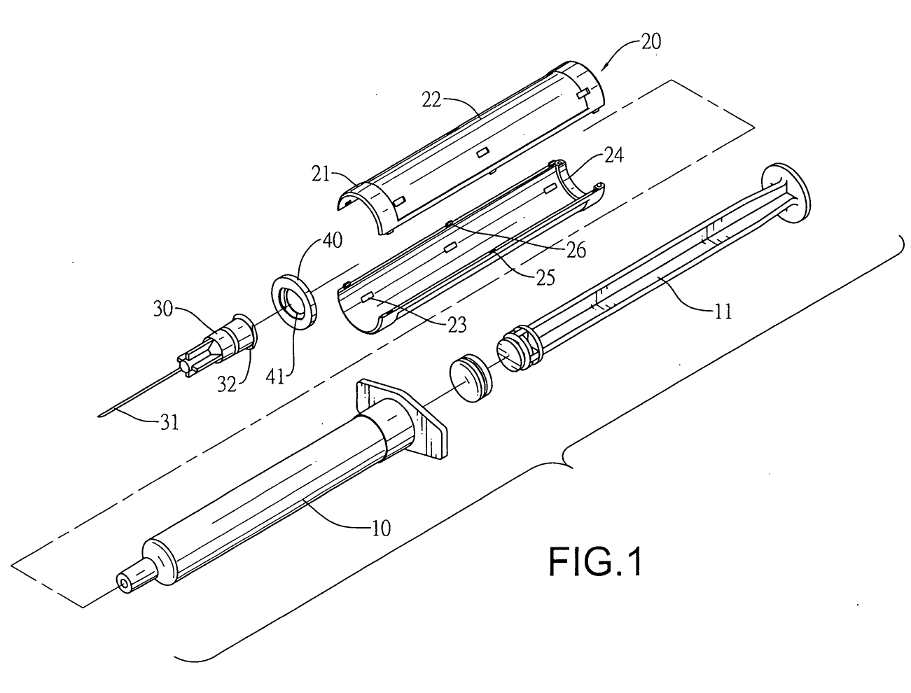

With further reference to FIGS. 1, 8, 10 and 14, the outer barrel (20)(20D) may be combined with at least two halves (21). Each half (21) has an inner surface, an outer surface, two sides, a pawl (24), multiple stoppers (23), multiple engaging bosses (26), multiple engaging recesses (25) and an outer recess (22).

The inner surface of each half (20)(20A) has a rear end.

The pawl (24) is formed at and radially extends from the rear end of the inner surface of the half (20)(20D).

The engaging bosses (26) and the engaging recesses (25) are formed at two sides of each half (21)(21D) and correspond respectively to the engaging bosses (26) and the engaging recesses (25) on the other half (21)(21D). When each engaging boss (26) selectively engages with the corresponding engaging recess (25), the at least two half (21)(21D) are combined to an outer barrel (20)(20D) that is able to attach to the inner barrel (10)(10A). The at least two pawls (24) are combined to an annular flange.

The outer recess (22) is formed at the out surface of each half (21)(21D). The outer recess (22) allow each half (21)(21D) to make in less material.

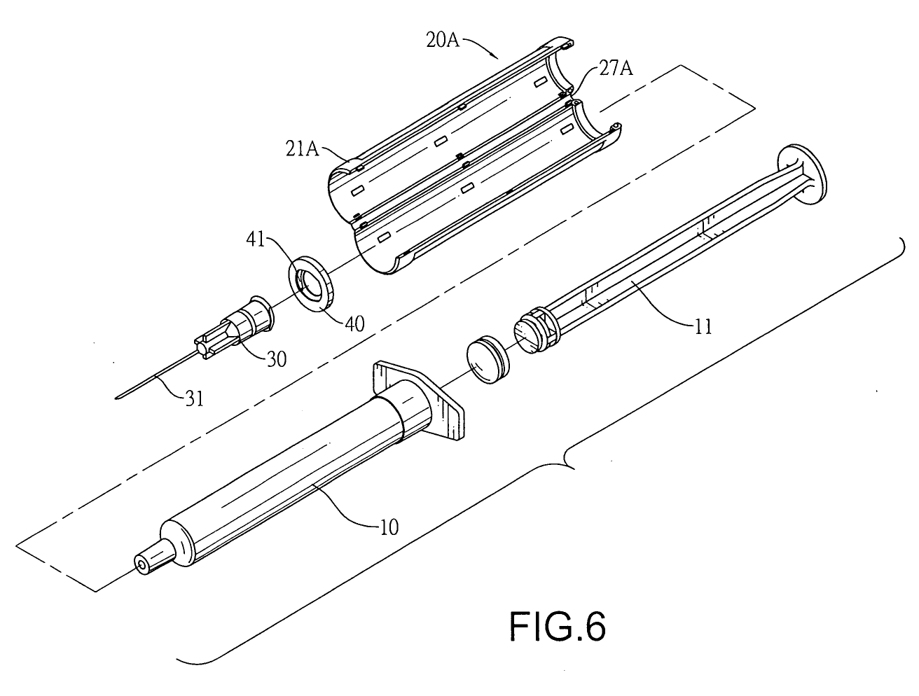

With further reference to FIG. 6, the outer barrel (20A) further has a connecting member (27A). The connecting member (27A) is mounted between and connects sides of two halves (21A) so that the halves (21A) are combined to an outer barrel (20A) with the connecting member (27A).

A disengaging device for a needle hub in accordance with the present invention comprises a seat (40)(40A)(40B)(40C)(40D)(40E).

With further reference to FIGS. 1, 6 and 7, a first embodiment of a disengaging device, the seat (40) is a ring and has an engaging edge, a side surface and an engaging recess (41). The engaging recess (41) is formed through the seat (40) and holds the annular shoulder (32) of the needle hub (30). The engaging edge of the seat (40) is formed around the seat (40) and abuts with the annular flange of the outer barrel (20)(20B).

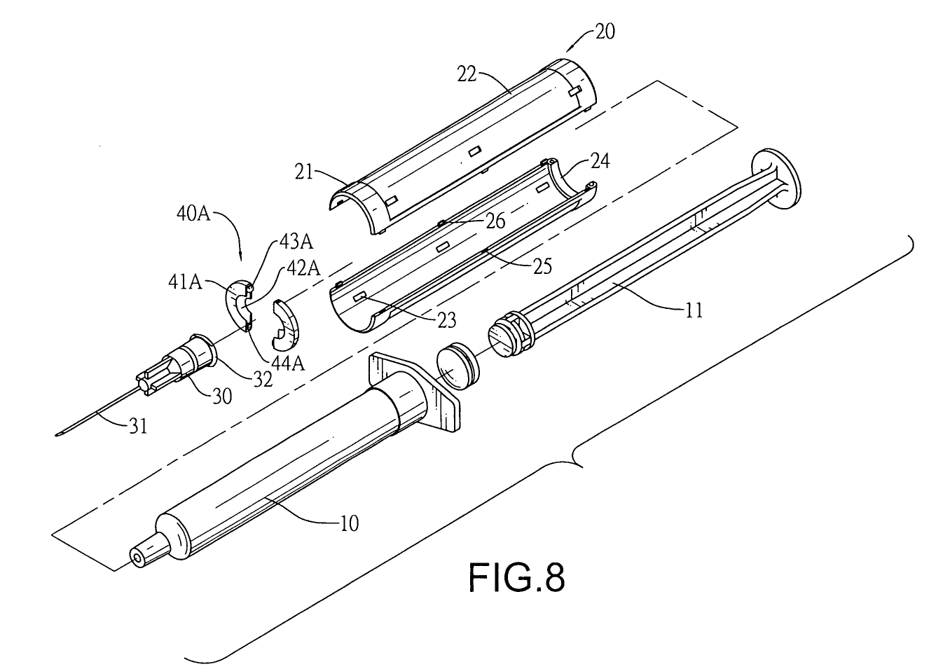

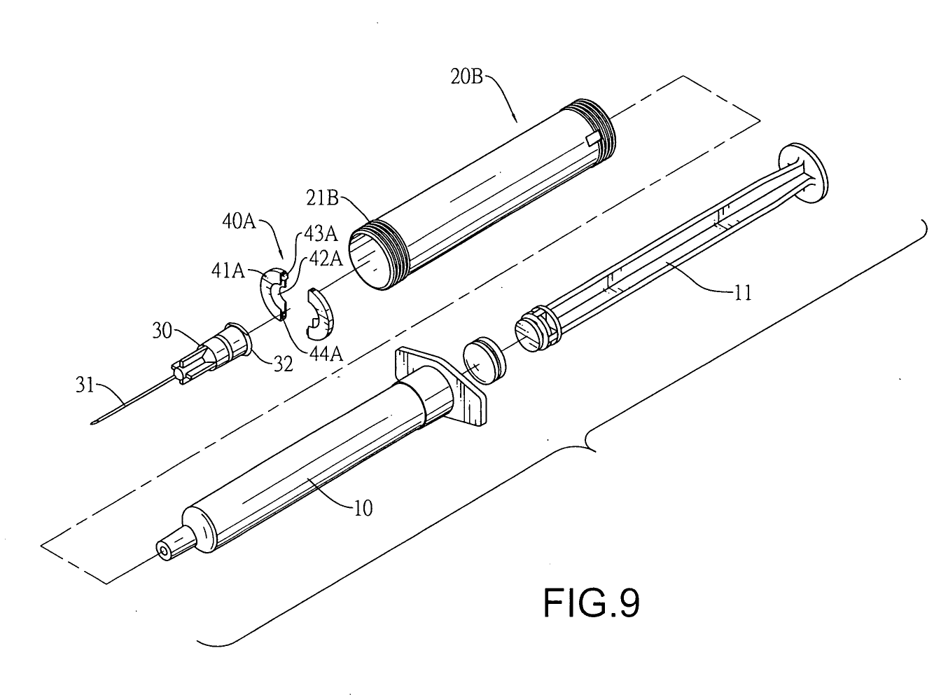

With further reference to FIGS. 8 and 9, a second embodiment of a disengaging device comprises a seat (40A) combined with at least two seat halves (41A). Each seat half (41A) has an inner side, an outer edge, a recess (42A), a boss (43A) and a boss recess (44A). The inner side has two ends. The recess (42A) is formed at inner side of the seat half (41A). The boss (43A) and the boss recess (44A) are formed respectively at the two ends of the inner side of the seat half (41A). When the boss (43A) engages with the boss recess (44A) in the other half (41A), the at least two seat halves (41A) are combined to a seat (40A) and the at least two recesses (41A) are combined to an engaging recess that receives the annular shoulder (32) of the needle hub (30). The at least two outer edges are combined to an engaging edge that abuts with the annular flange on the outer barrel (20)(20B).

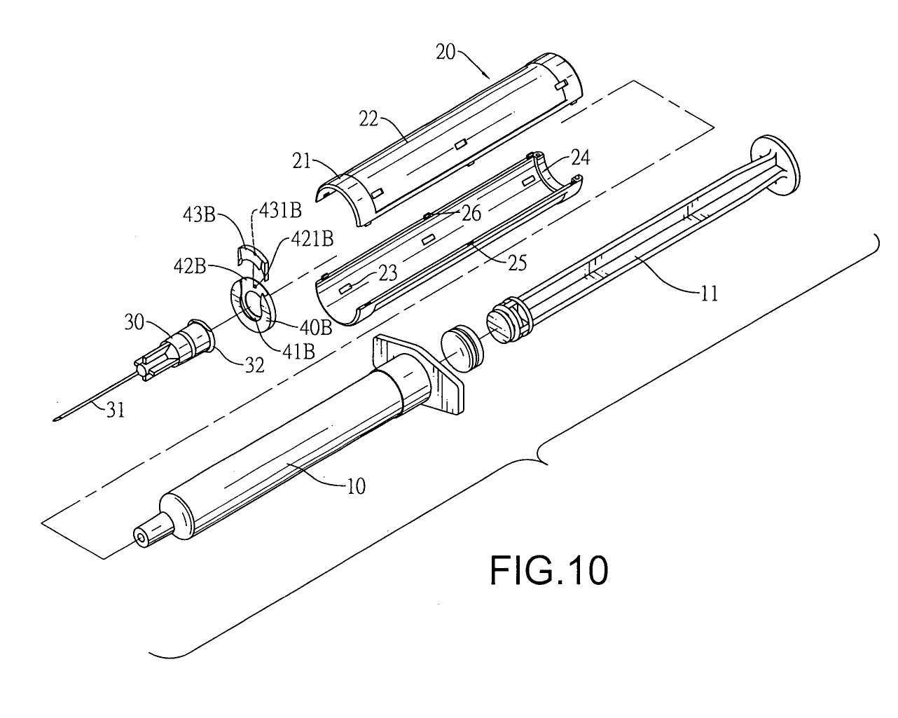

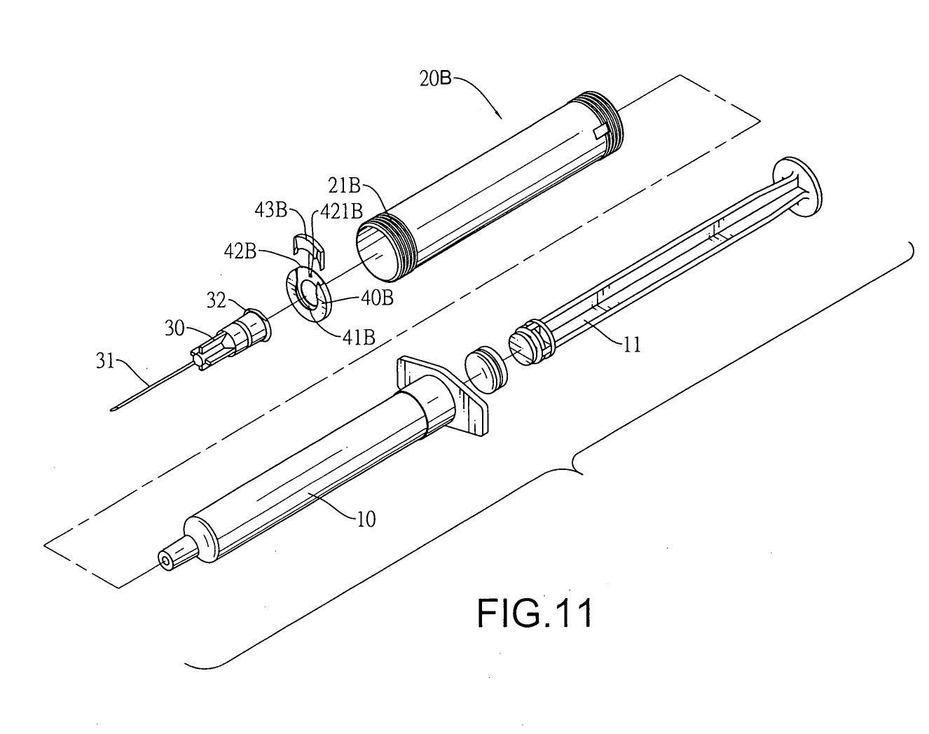

With further reference to FIGS. 10 and 11, a third embodiment of a disengaging device in accordance with the present invention comprises a seat (40B) having a side opening (42B) and a retaining tab (43B). The side opening (42B) is formed at the side surface of the seat (40B) and communicates with the engaging recess (41B) of the seat (40B) and has a bottom surface and an extension. The extension (421B) is formed at the bottom surface of the side opening (42B). The retaining tab (43B) has a bottom side and a recess (431B). The recess (43 1B) is formed at the bottom side of the retaining tab (43B). When the annular shoulder (32) of the needle hub (30) extends into the engaging recess (41B) from the side opening (42B), the retaining tab (43B) is put into the side opening (42B) of the seat (40B) and the extension (421B) of the side opening (42B) extends into and engages with the recess (43B) of the retaining tab (43B) so that the retaining tab (43B) closes the side opening (42B) to hold the annular shoulder (32) of the needle hub (30) inside the engaging recess (41B) in the seat (40B).

With further reference to FIG. 12, a fourth embodiment of a disengaging device in accordance with the present invention comprises a seat (40C) being cone-shaped and having a side, a front end, a rear end, a engaging recess (41C) and an engaging edge (42C). The engaging recess (41C) is formed on the front end of the seat (40C) and receives the annular shoulder (32A) of the needle hub (30A). The engaging edge (42C) is formed at rear end of the seat (40C) and abuts with the annular flange of the outer barrel (20C).

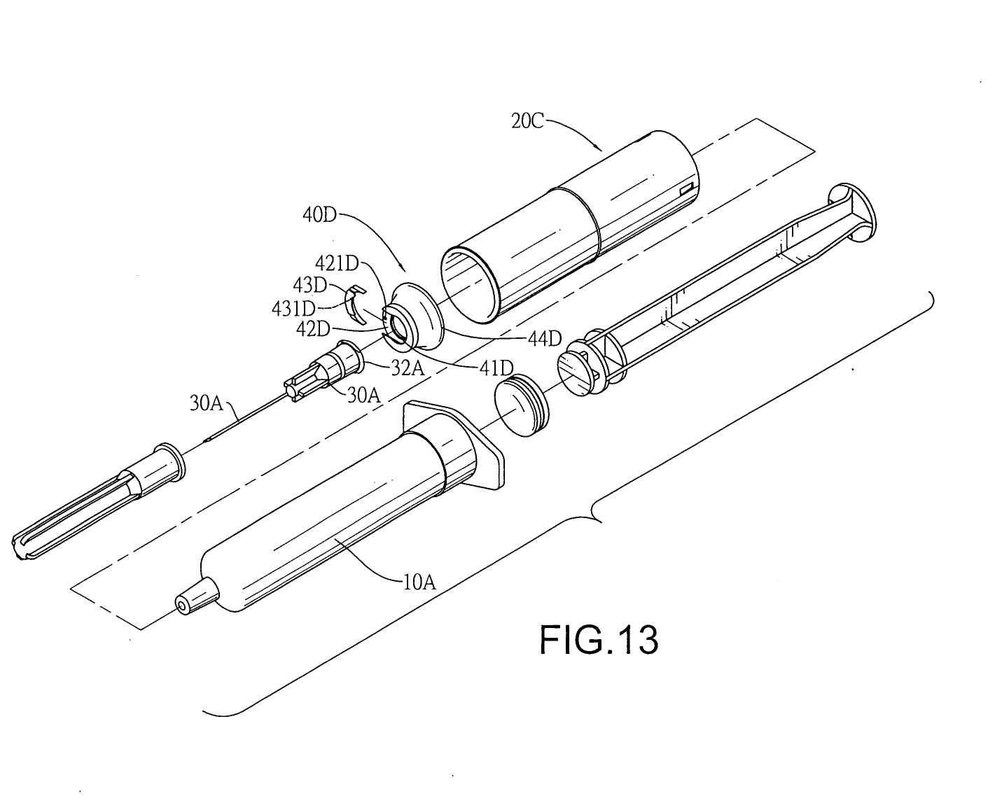

With further reference to FIG. 13, a fifth embodiment of a disengaging device in accordance with the present invention comprises a seat (40D) having a side opening (42D) and a retaining tab (43D). The side opening (42D) is formed at the side of the seat (40D), communicates with the engaging recess (41D) of the seat (40D) and has a bottom surface and an extension (421D). The extension (421D) is formed at the bottom surface of the side opening (42D). The retaining tab (43D) has a bottom side and a recess (431D). The recess (431D) is formed at the bottom side of the retaining tab (43D). When the annular shoulder (32A) of the needle hub (30A) extends into the engaging recess (41D) from the side opening (42D), the retaining tab (43D) is put into the side opening (42D) of the seat (40D) and the extension (421D) of the side opening (42D) extends into and engages with the recess (431D) of the retaining tab (43D) so that the retaining tab (43D) closes the side opening (42D) to hold the annular shoulder (32A) of the needle hub (30A) inside the engaging recess (41D) in the seat (40D).

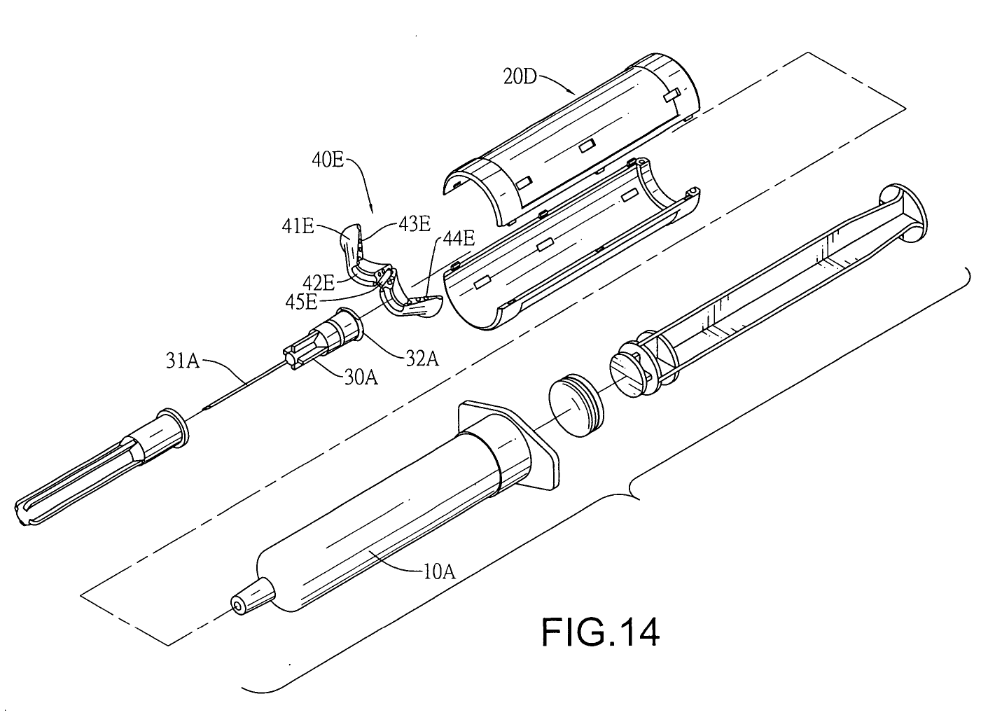

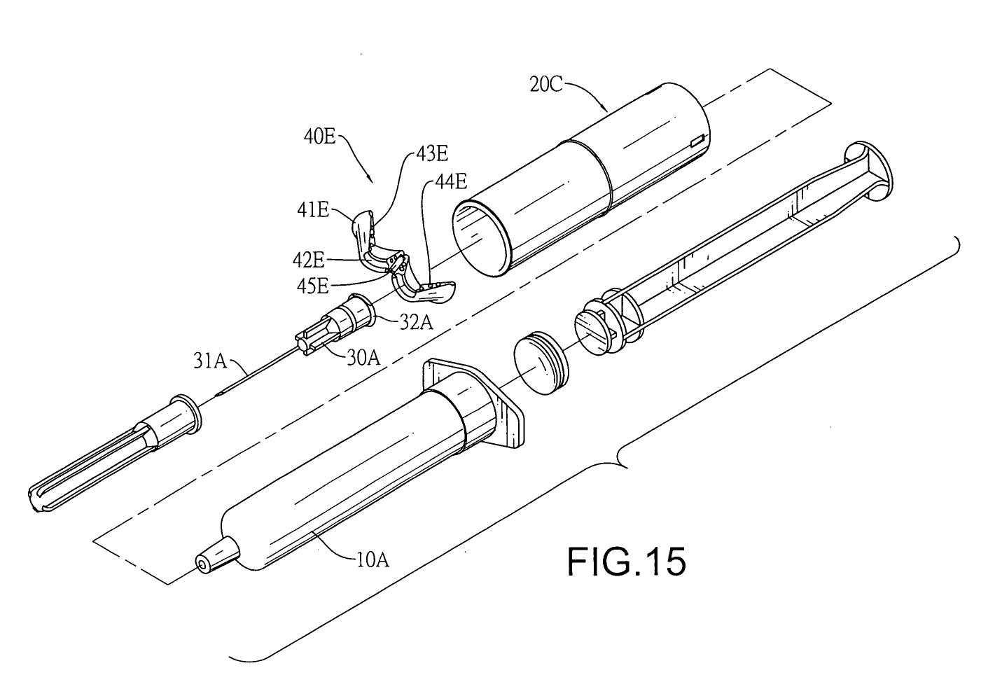

With further reference to FIGS. 14 and 15, a sixth embodiment of a disengaging device in accordance with the present invention comprises a seat (40E) being cone-shaped and having at least two seat halves (41E) and a connect member (45E). Each seat half (41E) has a front end, a rear end, an inner side, a front recess (42E), multiple boss recesses (43E) and multiple bosses (44E). The inner side has two ends. The front recess (42E) is formed at the inner side of the seat half (41E) near the front end. The bosses (44E) and the boss recesses (43E) are formed respectively at the two ends of the inner side of the seat half (41E). When the bosses (44E) selectively engage with the corresponding boss recesses (43E) in the other seat half (41E), the at least two halves (41E) are combined to a seat (40E) and at least two front recesses (42E) is combined to a engaging recess that receives the annular shoulder (32A) of the needle hub (30A). The at least two rear ends of the at least two seat halves (41E) are combined to an engaging edge that abuts with the annular flange of the outer barrel (20C)(20D). The connecting member (45E) is mounted between and connects two sides of two seat halves (41E) to combine the seat halves (41E) together.

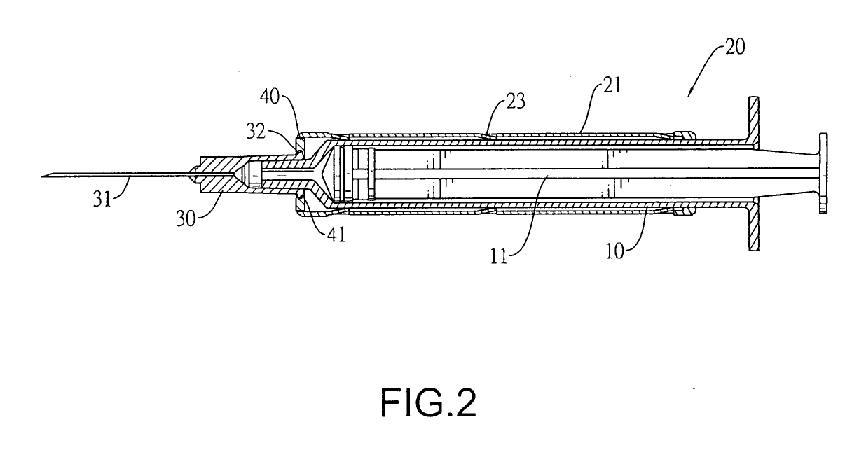

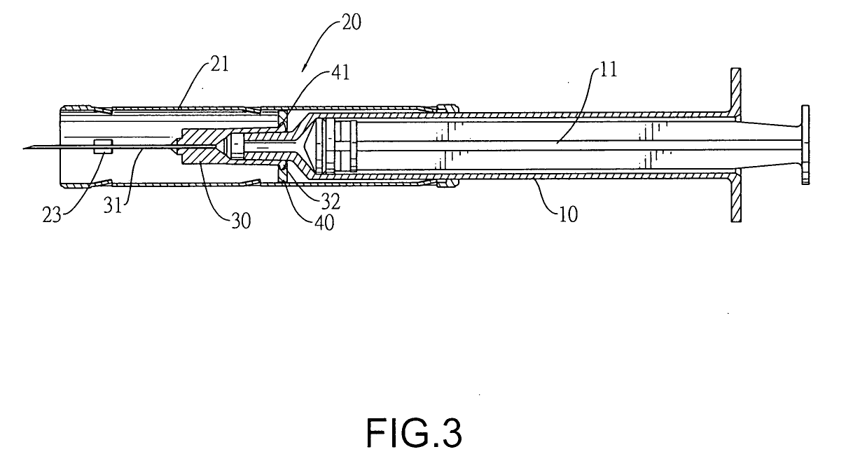

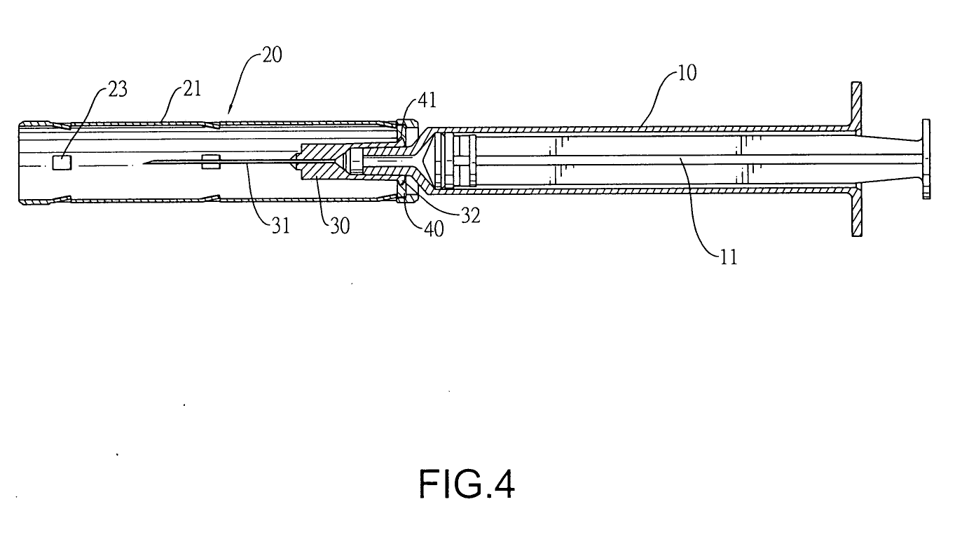

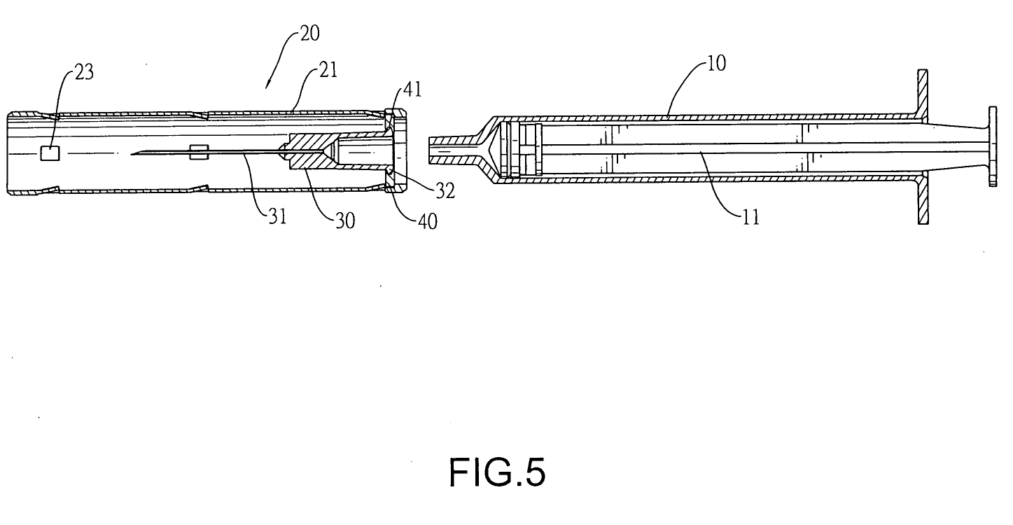

With further reference to FIGS. 2, 3, 4 and 5, after medication is injected into a patient, the inner barrel (10) is pulled out of the outer barrel (20). The seat (40) engages with the annular flange of the outer barrel (20). When the inner barrel (10) and the outer barrel (20) are separated, the needle hub (30) with the needle (31) is held inside the outer barrel (20) with the engagement between the annular flange and the engaging edge on the disengaging device. The needle (31) cannot be used again and cannot injure anyone when the needle (31) is held inside the outer barrel (20).

Consequently, because the seats (40)(40A)(40B)(40C)(40D)(40E) can be fit with different needle hub (30)(30A), to assemble different types of safety syringes with the seat (40)(40A)(40B)(40C)(40D)(40E) is easy and convenient.

Even though numerous characteristics and advantages of the present invention have been set forth in the foregoing description together with details of the structure and function of the invention, the disclosure is illustrative only. Changes may be made in detail especially in matters of shape, size, and arrangement of parts within the principles of the invention to the full extent indicated by the broad general meaning of the terms in which the appended claims are expressed.

What is claimed is:

1. A disengaging device for a needle hub of a safety syringe and having an annular shoulder formed at rear end of the needle hub, the safety syringe having an outer barrel having an annular flange formed at an inner surface of the outer barrel and an inner barrel being inserted into the outer barrel and having a front end allow the needle hub to mount thereon, and the disengaging device comprising:
a seat being adapted to be selectively mounted on the annular shoulder of the needle hub and having
an engaging recess formed at the seat and adapted for receiving the annular shoulder of the needle hub.

2. The disengaging device as claimed in claim 1, wherein the seat has an engaging edge formed around the seat and a side surface in which the engaging recess is defined.

3. The disengaging device as claimed in claim 2, wherein the seat is a ring.

4. The disengaging device as claimed in claim 3, wherein the seat further has
a side opening formed at the side surface of the seat and communicating with the engaging recess in the seat and having a bottom surface; and
a retaining tab mounted in and engaged with the side opening of the seat and having a bottom side.

5. The disengaging device as claimed in claim 4, wherein the side opening of the seat has an extension formed at the bottom surface of the side opening; and
the retaining tab of the seat has a recess formed at the bottom side of the retaining tab engaging with the recess of the retaining tab so that the retaining tab closes the side opening to hold the annular shoulder of the needle hub inside the engaging recess of the seat.

6. The disengaging device as claimed in claim 1, wherein the seat has at least two seat halves combined with each other, each seat half having an inner side, an outer edge, a recess formed at inner side of the seat half and the at least two recesses are combined to the engaging recess that is adapted for receiving the annular shoulder of the needle hub.

7. The disengaging device as claimed in claim 6, wherein each seat half has a boss formed at one of the two ends of the inner side of the seat half and a boss recess formed at one of the two ends of the inner side of the seat half and selectively engaging to the boss on the other seat half so that the at least two seat halves are combined to the seat.

8. The disengaging device as claimed in claim 1, wherein the seat has a side, a front end, a rear end, an engaging recess formed on the front end of the seat for receiving the annular shoulder of the needle hub and an engaging edge formed at the rear end of the seat to engage with the annular flange of the outer barrel.

9. The disengaging device as claimed in claim 8, wherein the seat further has a side opening formed at the side of the seat and communicating with the engaging recess and having a bottom surface and a retaining tab engaged with the side opening of the seat and having a bottom side.

10. The disengaging device as claimed in claim 9, wherein the side opening of the seat further has an extension formed at the bottom surface of the side opening; and
the retaining tab further has a recess formed at the bottom side of the retaining tab and engaging with the extension on the side opening so that the retaining tab closes the side opening to mount the annular shoulder of the needle hub into the engaging recess of the seat.

11. The disengaging device as claimed in claim 10, wherein the seat is cone-shaped.

12. The disengaging device as claimed in claim 1, wherein the seat has at least two seat halves combined with each other, each seat half having front end, a rear end, an inner side and a front recess formed at the inner side of each seat halves and near the front end and at least two front recesses being to combine into an inner recess that is able to receive the annular shoulder of the locking hub.

13. The disengaging device as claimed in claim 12, wherein each seat half has multiple boss recesses formed separately at the two ends of the inner side of the seat half and multiple bosses formed separately at the two ends of the inner side of the seat half and selectively engage to the boss recesses of the other half.

14. The disengaging device as claimed in claim 13, wherein the seat further has a connecting member mounted between two sides of two seat halves to combine the seat halves into the seat.

15. The disengaging device as claimed in claim 14, wherein the seat is cone-shaped.
